(19) United States
(12) Patent Application Publication
(10) Pub. No.: US 2019/0162672 A1
(43) Pub. Date: May 30, 2019

(54) IMAGE FORMING APPARATUS AND DETERMINATION METHOD

(71) Applicant: SHARP KABUSHIKI KAISHA, Sakai City, Osaka (JP)

(72) Inventor: YUSUKE SAKAKIBARA, Sakai City, Osaka (JP)

(73) Assignee: SHARP KABUSHIKI KAISHA, Sakai City, Osaka (JP)

(21) Appl. No.: 16/322,236

(22) PCT Filed: Mar. 14, 2017
(86) PCT No.: PCT/JP2017/010260

§ 371 Date: Jan. 31, 2019

(30) Foreign Application Priority Data
Aug. 10, 2016 (JP) 2016-158159

(51) Int. Cl. G01N 21/86; G01N 21/31; G01N 33/34

(52) U.S. Cl. CPC G01N 21/86; G01N 21/31; G01N 2021/3129; G01N 2021/8663; G01N 33/346

(57) ABSTRACT

Provided is an image forming apparatus having a simplified sensor structure and configured to determine both the moisture content and paper type of paper. An image forming apparatus includes light-emitting units configured to illuminate paper—with a plurality of types of emission light having mutually different peak wavelengths, a light-receiving unit configured to receive reflected light, reflected from the paper, of the plurality of types of emission light, and a paper determination unit configured to determine the moisture content and the paper type of the paper, based on received-light intensities of the plurality of types of reflected light.

### TECHNICAL FIELD

The following disclosure relates to an image forming apparatus configured to form an image on paper, and also relates to a paper determination method.

### BACKGROUND ART

Image forming apparatuses, such as copying machines, printers, fax machines, and multifunctional peripherals incorporating the functions of the above-mentioned machines, use various types of paper including woodfree paper, recycled paper, and coated paper. Even wet paper may be used in some environments.

To enhance the quality of images formed by an image forming apparatus, it is necessary to set various image forming conditions (e.g., transfer current, pressure applied at the time of fixing, fixing temperature, and fixing time) depending on the paper moisture content, the paper type, or the like. To this end, an image forming apparatus including a sensor for detecting the paper moisture content or the paper type has been developed.

PTL 1 describes an image forming apparatus including a sensor for calculating the moisture content from the amount of received light obtained by emitting, onto the paper (recording paper), light in a water absorption wavelength band, PTL 2 describes a sensor apparatus configured to determine the paper type of the paper (recording paper) by emitting light onto the paper and detecting the light from the paper with three photo detectors.

### CITATION LIST

### Patent Literature

PTL1: JP 2006-52069 A (published on Feb. 23, 2006)

PTL2: JP 2015-108611 A (published on Jun. 11, 2015)

### SUMMARY OF INVENTION

### Technical Problem

There has been no sensor capable of determining both paper moisture content and paper type. Hence, two types of sensors need to be separately attached to the apparatus so that both the paper moisture content and the paper type are simultaneously determined. The following describes problems caused by two types of sensors that are separately attached.

Those pieces of information acquired separately by the two types of sensors include some that are identical to each other, such as the characteristics of the light reflected from the paper. The two types of sensors, however, are developed such that their accuracies are independently enhanced. Hence, while using information acquired from one sensor, determination with the other sensor cannot be performed. Thus, the two types of sensors need to acquire the identical piece of information separately. This causes undesired increases in cost of sensors, the number of components, power consumption, and the space where sensors are attached.

In addition, acquiring the identical pieces of information twice increases the length of time needed for the measurement with the sensors. Meanwhile, in a case where the sensors are disposed in a paper-transport path in the image forming apparatus, for example, the image forming apparatus is required to measure the paper during a short length of time at which the paper is passing through the paper-transport path. Accordingly, a long measurement time may cause a big problem.

The following disclosure has been made in view of the above-described problems, and an object thereof is to provide an image forming apparatus having a simplified sensor structure and configured to determine both the moisture content and the paper type of paper.

### Solution to Problem

To solve the above-described problems, an image forming apparatus according to an aspect of the present invention includes: an illuminating unit configured to illuminate paper with a plurality of types of emission light having mutually different peak wavelengths; a light-receiving unit configured to receive a plurality of types of reflected light reflected from the paper, of the plurality of types of emission light; and a determination unit configured to determine the moisture content and the paper type of the paper, based on received-light intensities of the plurality of types of reflected light received by the light-receiving unit.

### Advantage Effects of Invention

The image forming apparatus according to an aspect of the present invention has a simplified sensor structure and is configured to determine both the moisture content and the paper type of the paper.

### BRIEF DESCRIPTION OF DRAWINGS

FIG. 1A is a schematic diagram illustrating a main part of an image forming apparatus according to a first embodiment. FIG. 1B is a bottom view of a sensor unit illustrated in FIG. 1A.

FIG. 2 is a block diagram illustrating a configuration of a main part of an image forming apparatus according to the first embodiment.

FIG. 3 is a graph showing straight lines represented by an equation (3) for different paper types.

FIG. 4 is a graph for describing thresholds used in paper-type determination.

FIG. 5 is a flowchart illustrating a flow of processes performed in an image forming apparatus of the first embodiment.

FIG. 6 is a flowchart illustrating a flow of processes performed in certain steps described in the flowchart of FIG. 5.

FIG. 7 is a block diagram illustrating a configuration of a main part of an image forming apparatus including a light-emission driving unit according to a modified example of the first embodiment.

FIG. 8 is a circuit diagram illustrating a light-emission driving unit and a main part of a sensor unit according to the modified example of the first embodiment.

FIG. 9A is a diagram illustrating a configuration of a main part of an image forming apparatus according to a second embodiment. FIG. 9B is a bottom view of a sensor unit illustrated in FIG. 9A.

FIG. 10 is a flowchart illustrating a flow of processes performed in an image forming apparatus of the second embodiment.

FIG. 11A is a diagram illustrating a configuration of a main part of an image forming apparatus according to a third embodiment. FIG. 11B is a bottom view of a sensor unit illustrated in FIG. 11A.

FIG. 12A is a diagram illustrating a configuration of a main part of an image forming apparatus according to a fourth embodiment. FIG. 12B is a bottom view of a sensor unit illustrated in FIG. 12A.

FIG. 13A is a diagram illustrating a configuration of a main part of an image forming apparatus according to a fifth embodiment. FIG. 13B is a bottom view of a sensor unit illustrated in FIG. 13A.

### DESCRIPTION OF EMBODIMENTS

### First Embodiment

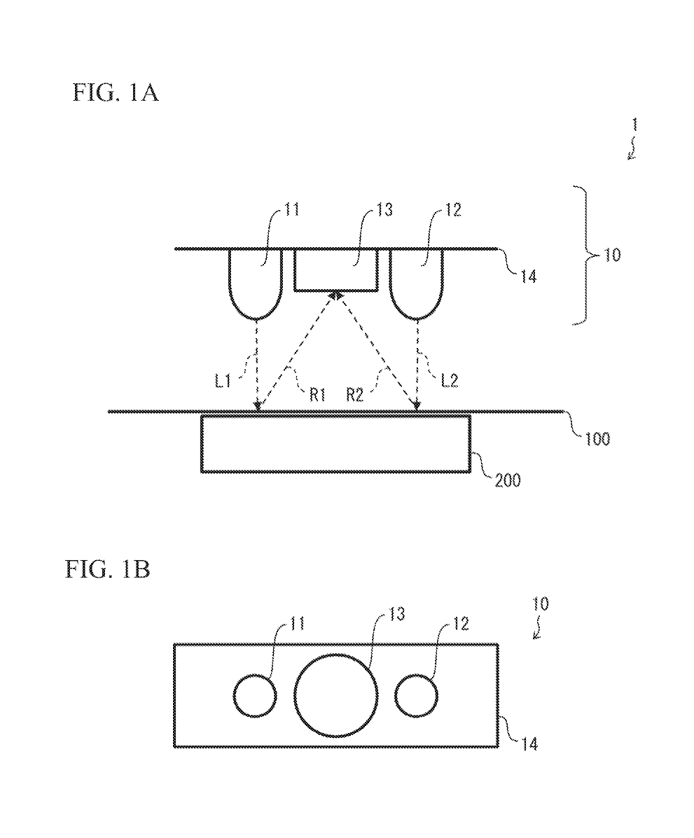

One embodiment of the present invention will be described below with reference to FIG. 1A to FIG. 8. In the present embodiment, a description is given of an image forming apparatus that is useful in a copying machine, a printer, a fax machine, and a multifunctional peripheral incorporating the functions of the above-mentioned machines. The image forming apparatus is configured to detect the paper type and the moisture content within a short length of time and to set the printing conditions.

FIG. 1A is a schematic diagram illustrating a main part of an image forming apparatus 1 according to the present embodiment. FIG. 1B is a bottom view of a sensor unit 10 illustrated in FIG. 1A. As illustrated in FIGS. 1A and 1B, the image forming apparatus 1 includes the sensor unit 10. In addition, as illustrated in FIG. 1A, in the image forming apparatus 1, paper 100 and a standard reflective plate 200 are disposed at a position facing the sensor unit 10.

The sensor unit 10 includes light-emitting units 11 and 12 (illuminating unit), a tight-receiving unit 13, and a substrate 14. The light-emitting units 11 and 12 illuminate the paper 100 or the like with multiple different types of emission light L1 and L2 having mutually different peak wavelengths. The peak wavelength of the emission light L1 differs from the peak wavelength of the emission light L2 by at least 10 nm. Hence, the image forming apparatus 1 is capable of determining the paper type and the moisture content of the paper 100 appropriately. Note that the value of 10 nm is the minimum value necessary for the determination of the paper type and the moisture content of the paper 100 in a case where an error in the peak wavelength of the LED is taken into consideration.

In the present embodiment, both of the light-emitting units 11 and 12 are Light Emitting Diodes (LEDs). The light-emitting units 11 and 12 may be any other light sources than LEDs such as laser light sources. In addition, it is preferable that the light emitted from the light-emitting units 11 and 12 have a narrow half-value angle.

The light-receiving unit 13 receives reflected light R1, R2, reflected from the paper 100 or the like, of the emission light L1, L2 emitted from the light-emitting units 11, 12. The light-receiving unit 13 is a single light-receiving sensor, and is a photodiode in the present embodiment. In the present embodiment, the light-receiving unit 13 is configured to receive a diffused light component of the light reflected from the paper 100 and the like. Note that an image forming apparatus according to an aspect of the present invention may include a plurality of light-receiving units.

The substrate 14 is a substrate to which the light-emitting units 11 and 12 as well as the light-receiving unit 13 are attached. To prevent the light emitted from the light-emitting units 11 and 12 from directly entering the light-receiving unit 13, it is preferable that a height of the light-receiving unit 13 relative to the substrate 14 be less than a height of each of the light-emitting units 11 and 12 relative to the substrate 14.

The sensor unit 10 may be attached, for example, in a paper-transport path through which the paper 100 is transported when the image forming apparatus 1 forms an image on the paper 100. Alternatively, the sensor unit 10 may be attached on, for example, a paper cassette configured to hold the paper 100 before the image forming apparatus 1 forms an image. Note that the sensor unit 10 is preferably mounted in the paper-transport path to reduce the time needed for the determination of the moisture content and the paper type of the paper 100.

The paper 100 is paper on which the image forming apparatus 1 forms an image. The paper 100 may be woodfree paper, recycled paper, coated paper, or the like.

The light reflected from the standard reflective plate 200 is used as the light indicating the reference received-light intensity that is used, by a controller 40 (to be described later), for determining the paper type. Specifically, the reflected light R1 and R2, reflected from the standard reflective plate 200, of the emission light L1, L2 is used as light having the reference received-light intensity. The material for the standard reflective plate 200 is not limited to a particular material. Any material may be used. The term “measurement” means that the light-receiving unit 13 acquires the received-light intensity of the reflected light R1, R2 reflected from the target object (e.g., the paper 100 or the standard reflective plate 200).

In the present embodiment, the standard reflective plate 200 is disposed in the paper-transport path through which the paper 100 is transported when the image forming apparatus 1 forms an image. The reflected light R1 and R2 from the standard reflective plate 200 is measured while no paper 100 is passing though the paper-transport path. Alternatively, the image forming apparatus 1 may be provided with a mechanism configured to switch the sensor unit 10 on and off so that the sensor unit 10 conducts the measurement for the standard reflective plate 200 in response to the standard reflective plate 200 moving to a measurement position of the sensor unit 10.

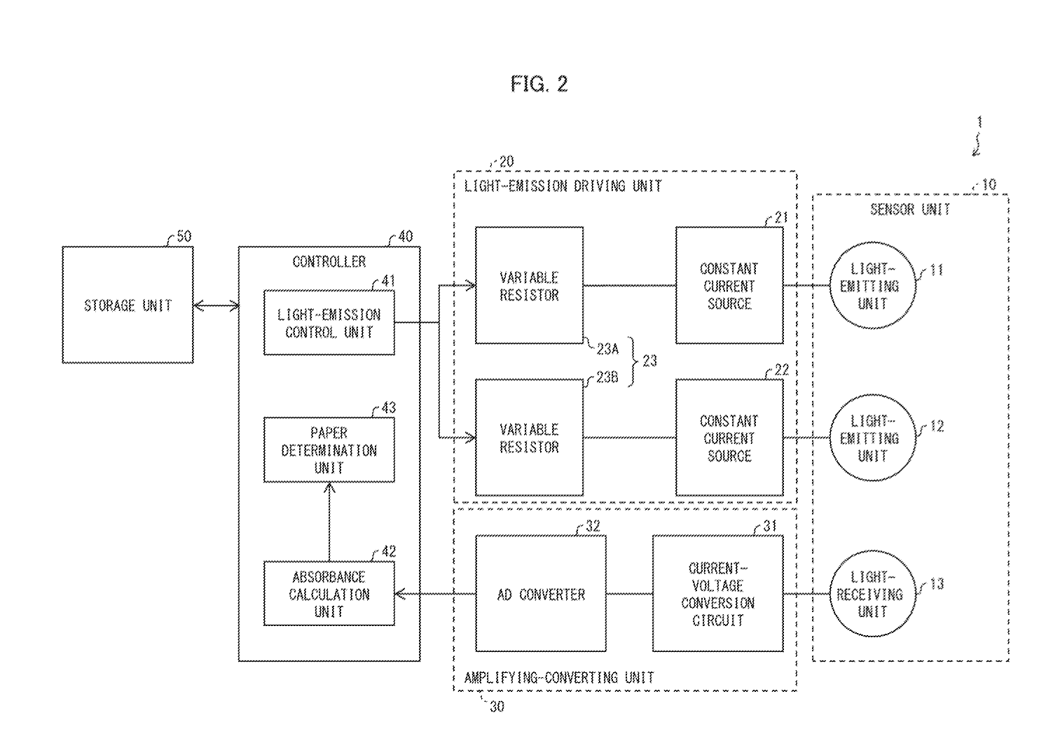

FIG. 2 is a block diagram illustrating a configuration of the main part of the image forming apparatus 1. As illustrated in FIG. 2, the image forming apparatus 1 includes the sensor unit 10, a light-emission driving unit 20, an amplifying-converting unit 30, a controller 40, and a storage unit 50. Since the sensor unit 10 is described above with reference to FIGS. 1A and 1B, no more description will be provided below.

The light-emission driving unit 20 is a driving circuit configured to drive the light-emitting units 11 and 12. The light-emission driving unit 20 includes: constant current sources 21 and 22 configured to supply electric power to the light-emitting units 11 and 12; and a variable resistor 23 whose resistance value is controllable by the controller 40 (to be described later). The constant current sources 21 and 22 can provide a current having a current value in accordance with the resistance value of the variable resistor 23. The variable resistor 23 includes, for example, two variable resistors 23A and 23B that correspond to the light-emitting units 11 and 12, respectively. The two variable resistors 23A and 23B are independently controlled by the controller 40 for a case of driving the light-emitting unit 11 and for a case of driving the light-emitting unit 12.

The amplifying-converting unit 30 is configured to amplify the output current from the light-receiving unit 13 corresponding to the intensity of the light received by the light-receiving unit 13 (received-light intensity) and convert a value of the amplified current into a digital value. The amplifying-converting unit 30 includes: a current-voltage conversion circuit 31 using an operational amplifier configured to amplify the output current; and an Analog-Digital (AD) converter 32 configured to convert, into a digital value, an analog value of the voltage outputted from the current-voltage conversion circuit 31.

The controller 40 is configured to control the light-emission driving unit 20 and to determine the moisture content and the paper type of the paper on the basis of a signal from the amplifying-converting unit 30. The controller 40 includes a light-emission control unit 41, an absorbance calculation unit 42 (calculation unit), and a paper determination unit 43 (determination unit). The controller 40 may be, for example, a microcomputer.

The light-emission control unit 41 is configured to control the resistance value of the variable resistor 23 as described above. The light-emission control unit 41 may be configured to control, in addition to the resistance value of the variable resistor 23, the light-emitting units 11 and 12 so that the light-emitting units 11 and 12 switch between the lighting-up state and the going-out state.

The absorbance calculation unit 42 is configured to calculate the absorbance of the paper 100 on the basis of the received-light intensities of the reflected light R1 and R2 indicated by the signal from the amplifying-converting unit 30. An absorbance A1 of the light from the light-emitting unit 11 and an absorbance A2 of the light from the light-emitting unit 12 are calculated by the following equations (1-1) and (1-2), respectively.

A1=log(V01/V1) (1-1)

A2=log(V02/V2) (1-2)

In the equations (1-1) and (1-2), V01 and V02 represent the received-light intensities of the reflected light R1 and R2, respectively, reflected by the standard reflective plate 200. In addition, V1 and V2 represent the received-light intensities of the reflected light R1 and R2, respectively, reflected from the paper 100. In addition, log means the common logarithm (logarithm to the base 10).

The received-tight intensity as used herein represents the difference between the intensity of the signal outputted from the light-receiving unit 13 in a case where the emission light L1 or L2 is emitted and the intensity of the signal outputted from the light-receiving unit 13 in a case where no emitted light is emitted. Assume, for example, that a signal intensity Vs1 represents the intensity of the signal for a case where the emission tight L1 is emitted, and a signal intensity Vn1 represents the intensity of the signal for a case where no such emitted light is emitted. Then, in a case where the measurement target is the standard reflective plate 200, V01=Vs1−Vn1. In addition, in a case where the measurement target is the paper 100, V1=Vs1−Vn1. Likewise, assume that a signal intensity Vs2 represents the intensity of the signal for a case where the emission light L2 is emitted and a signal intensity Vn2 represents the intensity of the signal for a case where no such emitted light is emitted. Then, in a case where the measurement target is the standard reflective plate 200, V02=Vs2−Vn2; whereas in a case where the measurement target is the paper 100, V2=Vs2−Vn2.

The paper determination unit 43 is configured to determine the moisture content and the paper type of the paper 100 on the basis of the absorbance A1 and the absorbance A2 calculated by the absorbance calculation unit 42. In the present embodiment, the paper determination unit 43 is configured to determine the moisture content and the paper type of the paper 100 by comparing a given straight line representing the changing absorbance depending on the moisture content for each type of paper with the absorbance A1 and with the absorbance A2. A specific determination method for determining the moisture content and the paper type of the paper 100 will be described below.

First, data on the absorbances A1 and A2 for the reflected light R1 and R2, for each type of paper that may be used in the image forming apparatus 1, are prepared beforehand by assuming various moisture contents Ms. The data may be stored as a database in the storage unit 50 (to be described later) by, for example, the manufacturer of the image forming apparatus 1. According to the Lambert-Beer law, the absorbances A1 and A2 change linearly with respect to the moisture content M. The following equations (2-1) and (2-2) representing straight lines on the A1-A2 plane are obtained by using a parameter M.

A1=a11×M+a10 (2-1)

A2=a21×M+a20 (2-2)

Equations (2-1) and (2-2) are prescribed mathematical expressions (prescribed functions) representing the changing absorbances A1 and A2 depending on the moisture content M for a prescribed paper type (paper X).

In the equations (2-1) and (2-2), a11, a10, a21, and a20 each represent constants each of which varies depending on the paper type and are stored in the database.

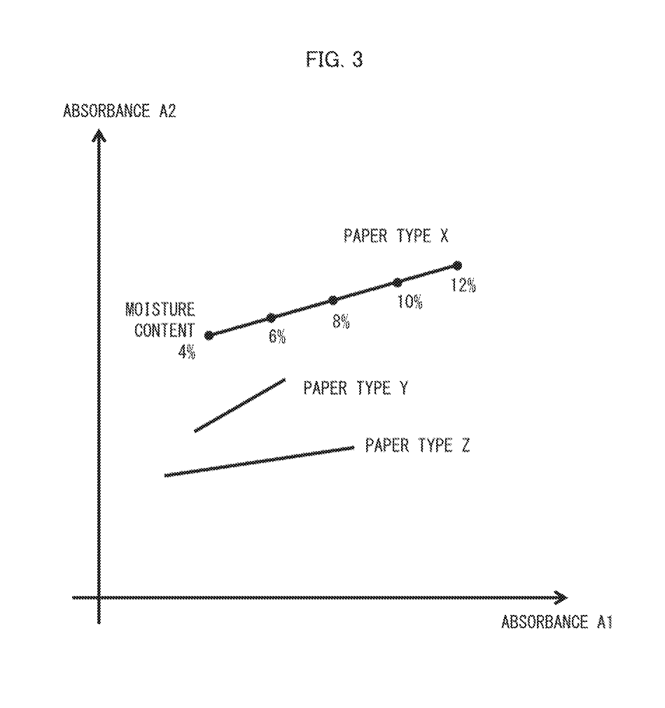

As shown in FIG. 3 (to be described later), the prescribed mathematical expressions representing the changing absorbances A1 and A2 depending on the moisture content M are prepared beforehand for three types of paper (paper type X, Y, and Z). As will be described below, the paper type and the moisture content of the paper 100 are determined by using these prescribed mathematical expressions.

In a case where the parameter M is eliminated from the equations (2-1) and (2-2), the obtained equation (3) described below is an equation of a straight line for the paper type X.

A2=ax×A1+bx (3)

In the equation (3), ax and bx are constants.

FIG. 3 is a graph showing straight lines expressed by the equation (3) for different paper types. In FIG. 3, the horizontal axis represents the absorbance A1 for the emission light L1, and the vertical axis represents the absorbance A2 for the emission light L2. FIG. 3 shows line segments of the straight lines for three paper types X, Y, and Z. The line segments correspond to the range of the moisture content M where the data exist. As shown by the line segments of paper type X, a point on the line segment for each of the paper types corresponds to a particular moisture content for the paper type.

The paper determination unit 43 is configured to calculate the distances lx, ly and lz between the point calculated by the absorbance calculation unit 42 and representing a set of the absorbances A1 and A2 (hereinafter, referred to as the “measurement point”) and the line segments for the paper types X, Y, and Z, respectively. Specifically, the paper determination unit 43 is configured to calculate the length of the perpendicular line from the measurement point to each of the line segments or to each of the straight lines including the line segments. Then, the paper determination unit 43 determines that the paper type corresponding to the line segment to which the distance from the measurement point is shortest is the paper type of the paper 100.

Note that a threshold lth of the distance that is used for the determination of the paper type is provided. In a case that all the distances lx to lz are greater than the threshold lth (i.e., lx>lth, ly>lth, and lz>lth), the paper determination unit 43 determines that the paper 100 is of a paper type not registered in the database and returns an error.

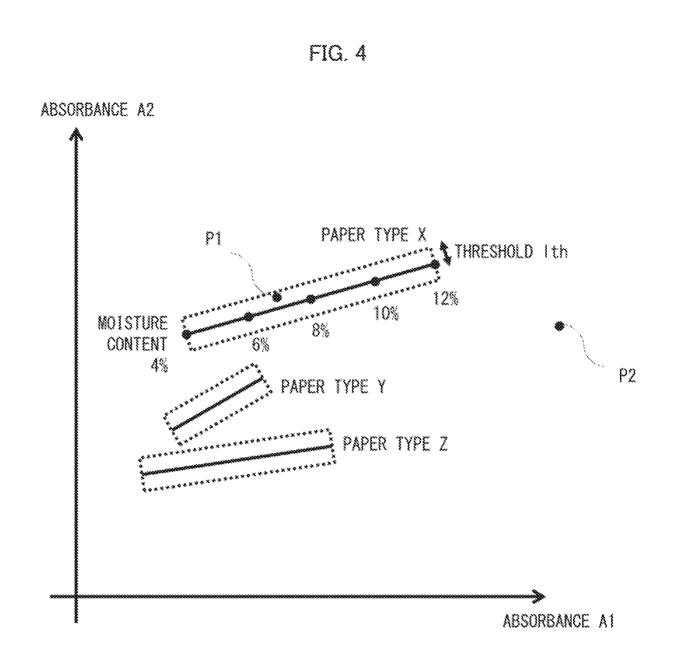

FIG. 4 is a drawing used for the description of the threshold lth. FIG. 4 is a graph obtained by adding, to the graph shown in FIG. 3, the ranges of the threshold lth corresponding to each of the paper types.

First, a description will be given of a case where the measurement point of the paper 100 is a point P1. As shown in FIG. 4, the point P1 exists within the range where a distance from the line segment corresponding to the paper types X is equal to or shorter than the threshold lth. Hence, in this case, the paper determination unit 43 determines that the paper 100 is of the paper type X.

Then, a description will be given of a case where the measurement point of the paper 100 is a point P2. As shown in FIG. 4, the point P2 has a larger distance from any of the line segments corresponding to the paper types X to Z than the threshold lth. Hence, in this case, the paper determination unit 43 determines that the paper 100 is none of the paper types X to Z registered in the database and returns an error.

The determination of the moisture content of the paper 100 is performed as follows. First, a perpendicular line from the measurement point of the paper 100 to the line segment or the straight line including the line segment corresponding to the determined paper type is drawn, and then the coordinates of the intersection point are calculated. Then, the value of the moisture content M can be determined by substituting the coordinates of the intersection points into any of the above-mentioned equations (2-1) and (2-2).

The controller 40 may be integrated, for example, with the sensor unit 10. Alternatively, the controller 40 may be incorporated, for example, into a circuit board configured to control the entire image forming apparatus 1.

The storage unit 50 is configured to store the data necessary for the processes performed by the controller 40. For example, the storage unit 50 stores the database for the above-mentioned paper types and the equations for the paper types.

### Process in Image Forming Apparatus 1

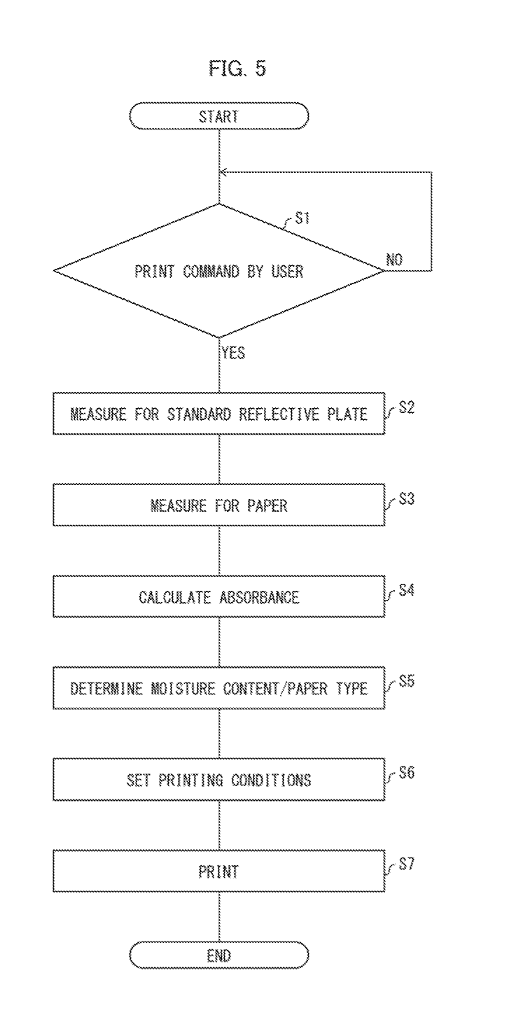

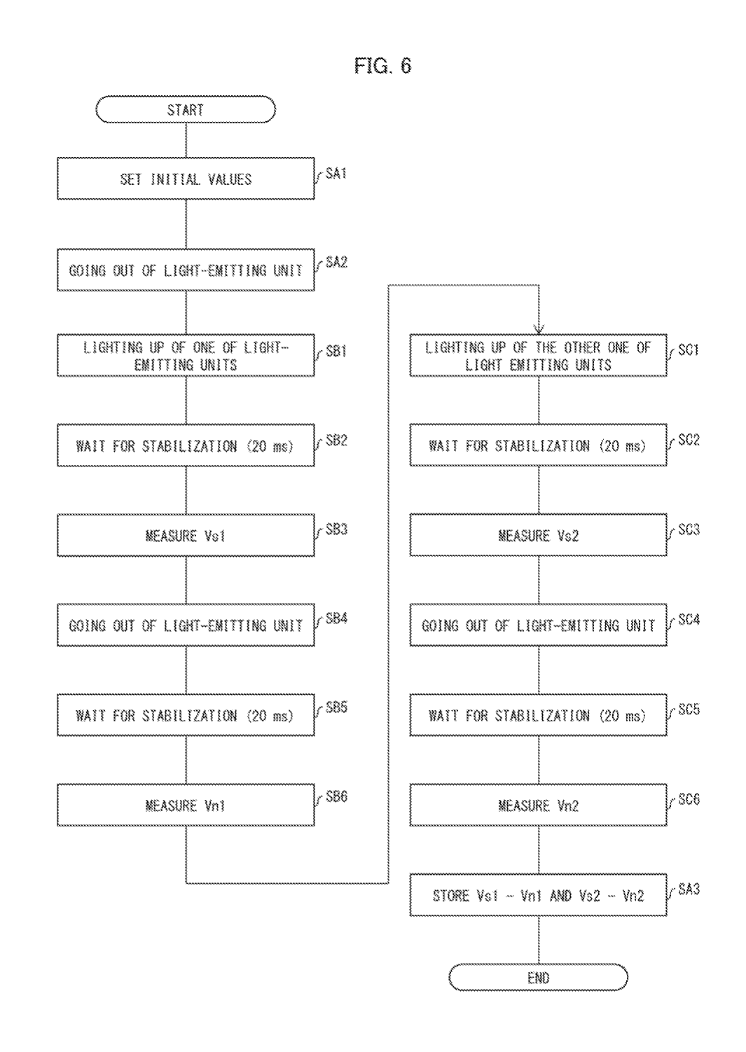

FIG. 5 is a flowchart illustrating processes performed in the image forming apparatus 1 of the present embodiment. First, the controller 40 waits for a print command by the user (S1). In a case where a print command is issued (YES in S1), the controller 40 causes the emission light L1 and L2 to be emitted onto the standard reflective plate 200, and thus the reflected light R1 and R2 is measured (S2). Then, the controller 40 causes the emission light L1 and L2 to be emitted onto the paper 100, and thus the reflected light R1 and R2 is measured (S3, light-emitting process, light-reception process). The steps S2 and S3 will be described later with reference to FIG. 6.

After the step S3, the absorbance calculation unit 42 calculates the absorbances of the paper 100 by using the equations (1-1) and (1-2) on the basis of the received-light intensities of the reflected light R1 and R2 reflected from the standard reflective plate 200 and the received-light intensities of the reflected light R1 and R2 reflected from the paper 100 (S4). Then, the paper determination unit 43 calculates the paper type and the moisture content of the paper 100 on the basis of the absorbances A1 and A2 of the paper 100 and the equations (2-1), (2-2), and (3) (S5, determination process).

Then, the controller 40 sets the image forming conditions (printing conditions) on the basis of the paper type and the moisture content of the paper 100 (determination result) (S6) and forms (prints) an image on the paper 100 (S7).

Examples of image forming conditions (printing conditions) set by the controller 40 include: the transfer current at the time of transferring the toner to the paper 100; the transport speed of the paper 100 at the time of fixing the toner onto the paper 100 (fixing time); the temperature of the heater rollers sandwiching the paper 100 (fixing temperature); and the pressure applied by the pressure roller (pressure applied at the time of fixing). For example, in a case where the paper 100 is of a paper type having a more irregular surface, the controller 40 increases the transfer current from the corresponding current for the paper 100 having a smooth surface and also increases the pressure applied at the time of fixing from the corresponding pressure for the paper 100 having a smooth surface. In addition, the controller 40 increases the fixing temperature or the fixing time for thick paper 100 from the corresponding temperature and time for thin paper 100. In addition, for example, the controller 40 decreases the transfer current for the paper 100 with a higher moisture content from the corresponding current for the paper 100 with a lower moisture content.

Note that in the flowchart illustrated in FIG. 5, the image forming apparatus 1 measures the received-light intensities of the reflected light R1 and R2 reflected from the standard reflective plate 200 and the paper 100 after the print command is issued by the user (YES in S1). Alternatively, the image forming apparatus 1 may measure the received-light intensities of the reflected light R1 and R2 reflected from the standard reflective plate 200 while waiting for the print command by the user; and may then measure the received-light intensities of the reflected light R1 and R2 reflected from the paper 100 after the reception of print command by the user. In a case where the sensor unit 10 is disposed in a cassette portion for the paper 100, the image forming apparatus 1 may measure the received-light intensities of the reflected light R1 and R2 reflected from both the standard reflective plate 200 and the paper 100 while waiting for the print command by the user.

FIG. 6 is a flowchart illustrating processes performed in steps S2 and S3 in the flowchart illustrated in FIG. 5. First, before the start of the measurement, the light-emission control unit 41 performs initial setting such as setting of the resistance value of the variable resistor 23 (SA1). Then, the light-emission control unit 41 switches off the light-emitting units 11 and 12 (SA2).

Then, the light-emission control unit 41 switches on only the light-emitting unit 11 (SB1), and waits for 20 ms until the light-emission state of the light-emitting unit 11 becomes stable and the output from the current-voltage conversion circuit 31 becomes constant (SB2). After that, the absorbance calculation unit 42 measures the signal intensity Vs1 of the signal outputted from the light-receiving unit 13 (SB3). The signal intensity Vs1 is a signal originally outputted from the light-receiving unit 13 and then amplified and converted into a digital value by the amplifying-converting unit 30.

Then, the light-emission control unit 41 switches off the light-emitting unit 11 (SB4), and waits for 20 ms until the light-emission state of the light-emitting unit 11 becomes stable and the output from the current-voltage conversion circuit 31 becomes constant (SB5). Then, the absorbance calculation unit 42 measures the signal intensity Vn1 corresponding to the background outputted form the light-receiving unit 13 (SB6).

Steps SB1 to SB6 are the steps of measuring the reflected light R1 of the emission light L1 emitted from the light-emitting unit 11. Likewise, the measurement is performed for the reflected light R2 of the emission light L2 emitted from the light-emitting unit 12 (SC1 to SC6). Hence, the signal intensity Vs2 of a case where the light-emitting unit 12 is emitting light and the signal intensity Vn2 corresponding to the background are measured. After that, the absorbance calculation unit 42 stores values resulting from Vs1−Vn1 and Vs2−Vn2 in the storage unit 50 (SA3).

Note that the waiting time at SB2, SB5, or the like until the light-emitting units 11 and 12 becomes stable and until the output of the current-voltage conversion circuit 31 becomes constant may be varied as appropriate depending on the specs of the light-emitting units 11 and 12, the specs of the current-voltage conversion circuit 31, or the like.

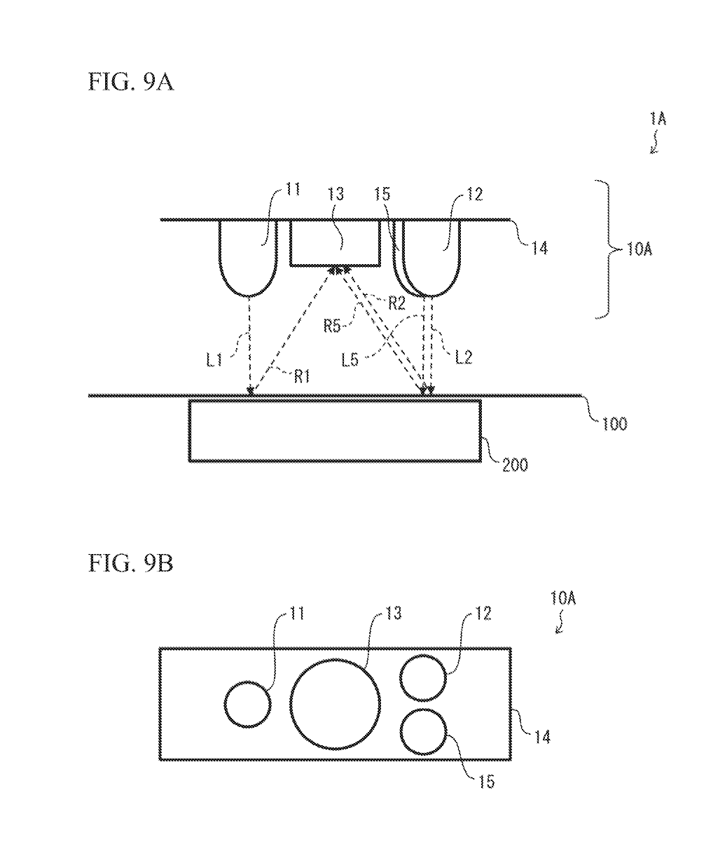

Note that the image forming apparatus 1 may include three or more light-emitting units (see, for example, FIGS. 9A and 9B described below). In a case where the light-emitting unit, as a whole, is capable of emitting two or more different kinds of light having mutually different peak wavelengths, two or more light-emitting units configured to emit the light having the same peak wavelength may be provided. Alternatively, the three or more light-emitting units may be configured to emit light having mutually different peak wavelengths from one another. In addition, no limits are imposed to the positions of the light-emitting units. One part of the light-emitting units and another part thereof may face each other with the light-receiving unit 13 interposed therebetween. Alternatively, all the light-emitting units may be disposed on the same side of the light-receiving unit 13.

In a case where the image forming apparatus 1 includes three or more light-emitting units, all the light-emitting units are connected to the light-emission driving unit 20, and are controlled so that all the light-emitting units that emit light having the same peak wavelength can be driven to emit light simultaneously. At S2 and S3, for each type of light emitted by the light-emitting unit, the controller 40 performs processes similar to the corresponding processes in SB1 to SB6 (or SC1 to SC6).

Furthermore, at S4, the absorbance calculation unit 42 calculates the absorbance for the different kinds of light. In a case where the number of the different types of light having mutually different peak wavelengths is n, the absorbance calculation unit 42 calculates n absorbances A1, A2, . . . , An.

Then, at S5, the paper determination unit 43 may determine the paper type and the moisture content of the paper, in a similar manner to a case of using only two types of light, by using the straight lines for the paper types and the points corresponding to the absorbances A1 to An for the paper 100 in the n-dimensional space.

Note that to determine the paper type of the paper 100, not only the above-described method using the prescribed mathematical equations, but prescribed data or a table (prescribed function) other than the mathematical equations may be used.

### Advantageous Effects of Image Forming Apparatus 1

The image forming apparatus 1 is capable of determining both the paper type and the moisture content of the paper 100 on the basis of the received-light intensity of the reflected light R1 and R2 received by the light-receiving unit 13. This simplifies the sensor structure, and both the paper type and the paper moisture content can also be determined. For example, even in a case where only a single light-receiving unit 13 is provided (where the sensor structure is especially simplified), both the paper type and the moisture content of the paper can be determined.

The use of the ratio of the received-light intensities of the reflected light R1 and R2 reflected from the paper 100 to the reference received-light intensity allows the influences by the error in the amount of light emitted from the light-emitting units 11 and 12 and in the amplification rate of the received-light signal to be eliminated and thus the measurement to be more accurate. In addition, the paper determination unit 43 can determine the paper type and the moisture content more easily than a case of using the common regression technique requiring, for example, the creation of a calibration curve of the moisture content, or than a common determination technique.

### Modified Example

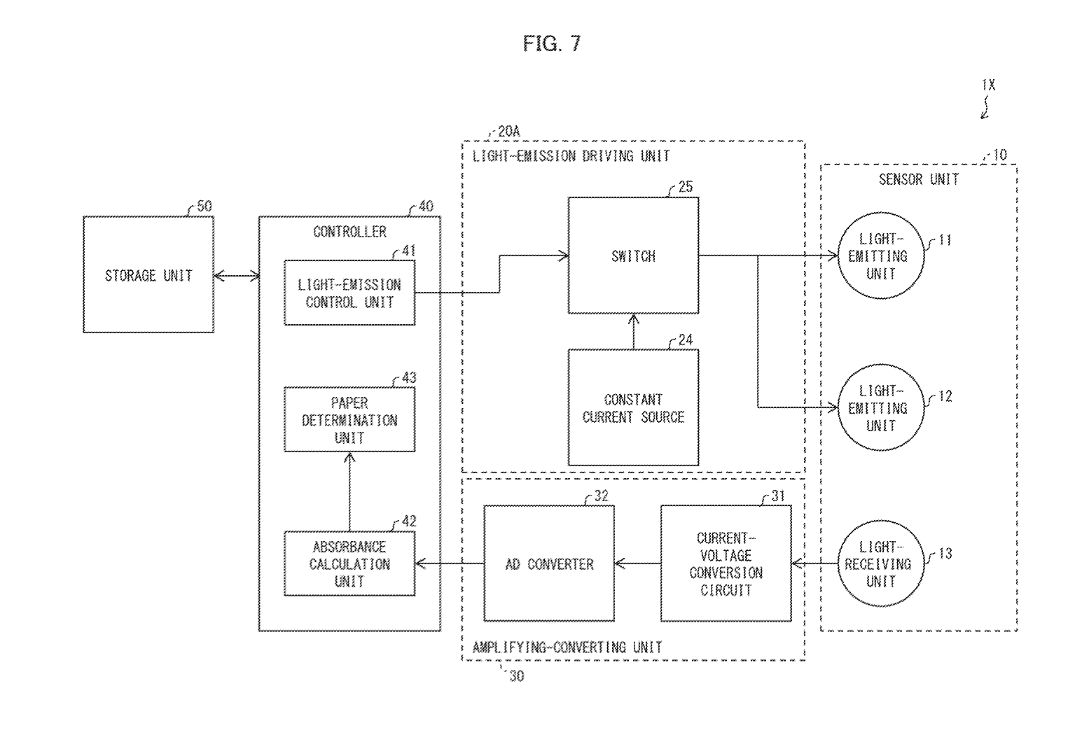

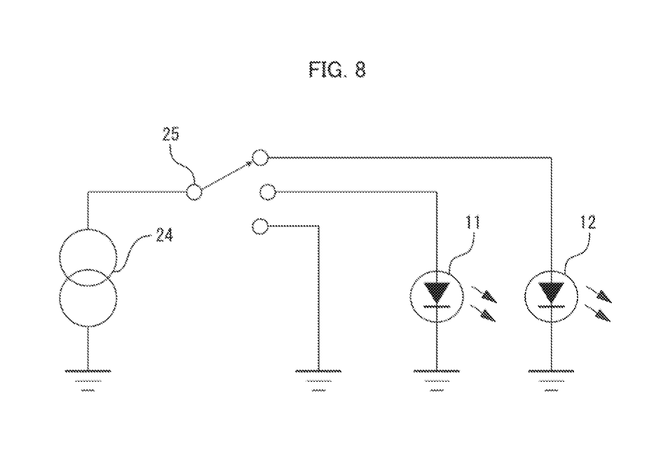

A modified example of the light-emission driving unit 20 will be described below with reference to FIG. 7 and FIG. 8. FIG. 7 is a block diagram illustrating a configuration of a main part of an image forming apparatus 1X including a light-emission driving unit 20A according to the modified example. FIG. 8 is a circuit diagram illustrating the light-emission driving unit 20A and a main part of a sensor unit 10.

As illustrated in FIG. 7, the light-emission driving unit 20A includes a constant current source 24 and a switch 25. The constant current source 24 is the single current source configured to supply electric power to the light-emitting units 11 and 12. The switch 25 is a tripolar switch configured to switch the state of connection of the constant current source 24 with the light-emitting units 11 and 12. The switch 25 may a relay that is controlled by, for example, the controller 40.

Specifically, as illustrated in FIG. 8, the switch 25 switches a state among the states (1) to (3).

(1) Going out (The constant current source 24 is connected to none of the light-emitting units 11 and 12.)

(2) Lighting up of the light-emitting unit 11 (The constant current source 24 is connected to the light-emitting unit 11.)

(3) Lighting up of the light-emitting unit 12 (The constant current source 24 is connected to the light-emitting unit 12.)

The image forming apparatus 1X including such a light-emission driving unit 20A can also determine both the paper type and the moisture content of the paper on the basis of the received-light intensities measured by the single light-receiving unit 13.

### Second Embodiment

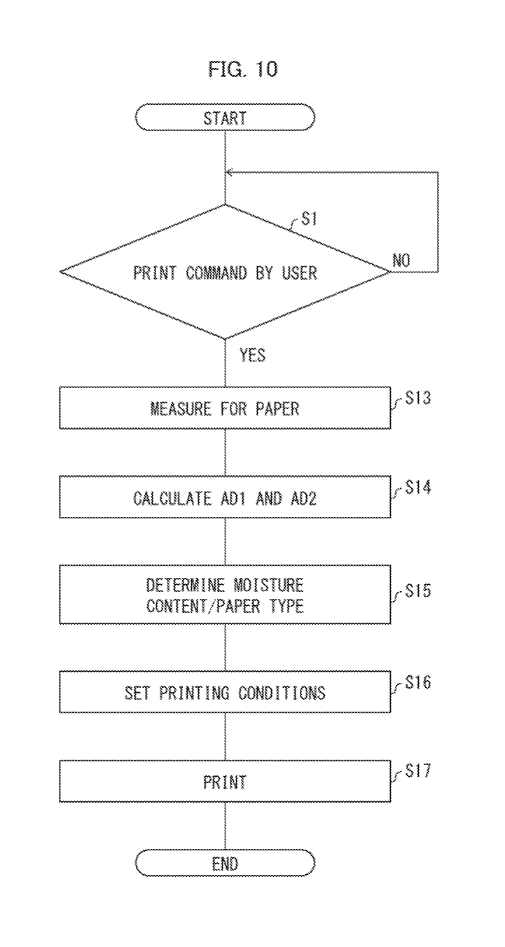

Another embodiment of the present invention will be described below with reference to FIG. 9A to FIG. 10. Note that, for convenience of description, components described in the respective embodiments are designated by the same reference numerals as those having the same function, and the descriptions of these components will be omitted.

FIG. 9A is a diagram illustrating a configuration of a main part of an image forming apparatus 1A according to the present embodiment. FIG. 9B is a bottom view of a sensor unit 10A illustrated in FIG. 9A. As illustrated in FIG. 9A and FIG. 9B, the sensor unit 10A includes a light-emitting unit 15 in addition to the components of the sensor unit 10. The light-emitting unit 15 is configured to emit emission light L5 having a peak wavelength that is different from any of the corresponding peak wavelengths of the light-emitting units 11 and 12.

The absorbance calculation unit 42 of the present embodiment is configured to calculate the absorbance of the paper 100 on the basis of the ratio between received-light intensities of the reflected light R1 and R2 reflected from the paper 100. Specifically, the absorbance calculation unit 42 is configured to calculate the absorbance of the paper 100 on the basis of the ratio between: the received-light intensities of the reflected light R1 and R2 of the single type of emission light L5 reflected from the paper 100; and the received-light intensities of the reflected light R1 and R2 of the other emission light L1 and L2 reflected from the paper 100. A specific determination method for determining the moisture content and the paper type of the paper 100 will be described below for a case of using three types of light having mutually different peak wavelengths.

First, in a case of using three kinds of light, the following equation (4) related to the absorbance A3 for the light from the light-emitting unit 15 can be obtained in addition to the equations (2-1) and (2-2) described above.

A3=a31×M+a30 (4)

In the equation (4), a31 and a30 represent constants each of which varies depending on the paper types.

By subtracting the equation (4) from each of the equations (2-1) and (2-2), the following equations (5-1) and (5-2) are obtained.

A1−A3=(a11−a31)×M+(a10−a30) (5-1)

A2−A3=(a21−a31)×M+(a20−a30) (5-2)

The equations (5-1) and (5-2) are subjected to the replacing processes of the following equations (6-1) to (6-6).

AD1=A1−A3 (6-1)

AD2=A2−A3 (6-2)

aD11=a11−a31 (6-3)

aD10=a10−a30 (6-4)

aD21=a21−a31 (6-5)

aD20=a20−a30 (6-6)

The following equations (7-1) and (7-2) can be obtained from the equations (5-1) and (5-2), respectively, by the replacing processes of the equations (6-1) to (6-6).

AD1=aD11×M+aD10 (7-1)

AD2=aD21×M+aD20 (7-2)

In a case where the equations (7-1) and (7-2) for all the paper types that may be used in the image forming apparatus 1A are stored in the database, the paper type and the moisture content of the paper can be determined on the AD1-AD2 plane by using a method similar to the method described in the first embodiment.

Note that the absorbance A3 of the light from the light-emitting unit 15 is calculated by the following equation (8).

A3=log(V03/V3) (8)

In the equation (8), V03 represents the received-light intensity of the reflected light R5 reflected from the standard reflective plate 200. In addition, V3 represents the received-light intensity of the reflected light R5 reflected from the paper 100.

The AD1 and AD2 can be obtained by the following equations (9-1) and (9-2).

AD1=A1−A3=log(V3/V1)+log(V01/V03) (9-1)

AD2=A2−A3=log(V3/V2)+log(V02/V03) (9-2)

The second term of each of the equations (9-1) and (9-2) is a constant determined not by the received-light intensity of the reflected light reflected from the paper 100, but solely by the received-light intensity of the reflected light reflected from the standard reflective plate 200. Hence, the value of the second term of each of the equations (9-1) and (9-2) can be measured and stored in the database beforehand at the time of manufacturing the image forming apparatus 1. In this case, the only things needed to obtain AD1 and AD2 are the measurements of the received-light intensities V1, V2, and V3 of the reflected light R1, R2, and R5, reflected from the paper 100, of the emission light L1, L2, and L5 emitted by the light-emitting units 11, 12, and 15. To put it differently, it is not necessary to measure the received-light intensities V01, V02, and V03 of the reflected light R1, R2, and R5 reflected from the standard reflective plate 200 every time the user issues a print command.

FIG. 10 is a flowchart illustrating the flow of processes performed in the image forming apparatus 1A of the present embodiment. The image forming apparatus 1A performs processes S13 and S14 in place of the processes S2 to S4 in the case of the image forming apparatus 1. Processes S15 to S17 in a case of the image forming apparatus 1A are similar to the processes S5 to S7 in the case of the image forming apparatus 1.

In a case that the result of the decision in S1 is YES, the controller 40 performs a measurement for the paper 100 (S13). At S13, as described above, the measurement is performed by using the emission light L1, L2, and L5 emitted by the light-emitting units 11, 12, and 15. Then, the absorbance calculation unit 42 calculates the absorbance of the paper 100 on the basis of the received-light intensities of the reflected light reflected from the paper 100 (S14).

As illustrated in FIG. 10, the image forming apparatus 1A only needs to perform measurement for the paper 100 (see FIGS. 9A and 9B) and does not need to perform measurement for the standard reflective plate 200. Hence, the moisture content and the paper type of the paper 100 can be determined by measuring the received-light intensities solely for the paper 100. Also in the image forming apparatus 1A, the use of the single light-receiving unit 13 and the current-voltage conversion circuit 31 allows the errors in the sensitivity of the light-receiving unit 13 and in the amplifying rate of the current-voltage conversion circuit 31 to be eliminated and thus the measurement to be done with higher accuracy.

### Third Embodiment

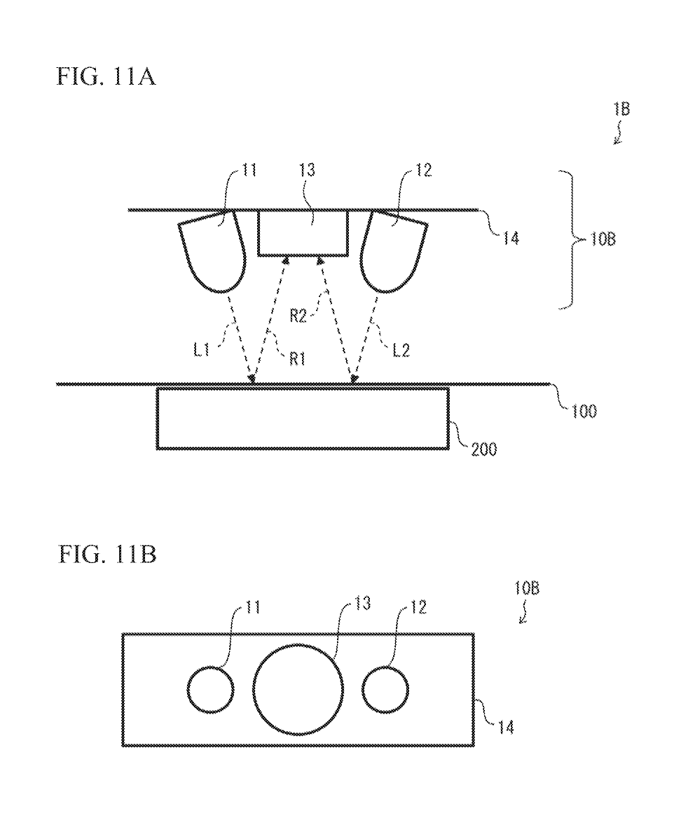

Another embodiment of the present invention will be described below with reference to FIGS. 11A and 11B.

FIG. 11A is a diagram illustrating a configuration of a main part of an image forming apparatus 1B according to the present embodiment. FIG. 11B is a bottom view of a sensor unit 10B illustrated in FIG. 11A. As illustrated in FIG. 11A and FIG. 11B, the sensor unit 10B differs from the sensor unit 10 in that the optical axes of the emission light L1 and L2 emitted by the light-emitting units 11 and 12 are not perpendicular to the paper 100. As illustrated in FIG. 11A, in the sensor unit 10B, the light-receiving unit 13 is configured to receive the reflected light R1 and R2, reflected regularly from the paper 100 (or from the standard reflective plate 200), of the components of the emission light L1 and L2 emitted in the optical-axis directions. Hence, in a case where the paper 100 is a paper that generates reflected light with a large regularly-reflected component (e.g., glossy paper), the light-receiving unit 13 can receive the reflected light R1 and R2 efficiently.

In the sensor unit 10B of the present embodiment, a normal line to the light-receiving surface of the light-receiving unit 13 may make a not-perpendicular angle with the paper 100. Specifically, it is preferable that the normal line be in parallel with the optical axes of the reflected light R1 and R2 incident on the light-receiving unit 13. Also in such a case, the light-receiving unit 13 can receive efficiently the reflected light R1 and R2 reflected from the paper 100.

### Fourth Embodiment

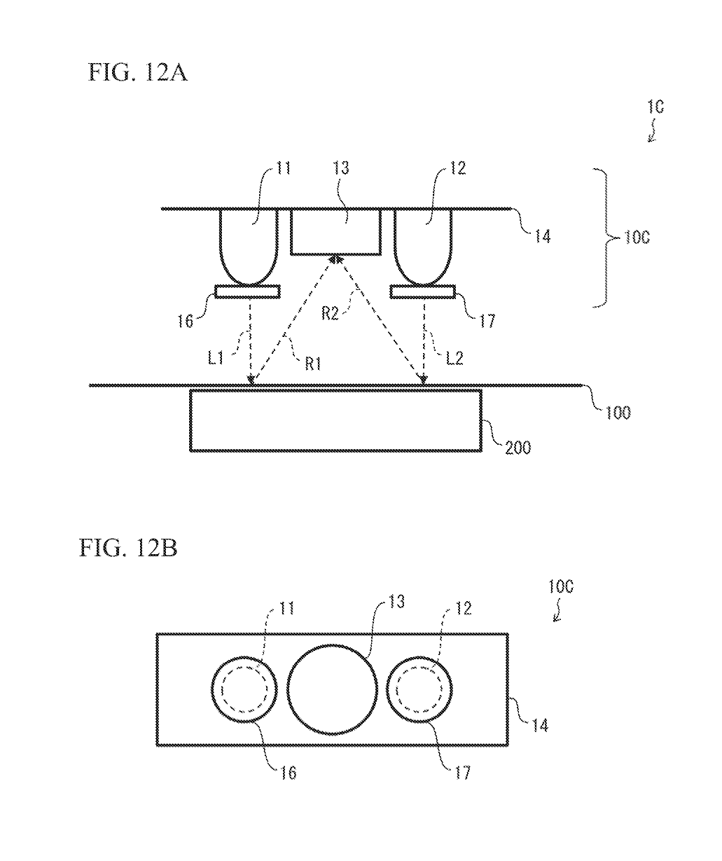

Another embodiment of the present invention will be described below with reference to FIGS. 12A and 12B.

FIG. 12A is a diagram illustrating a configuration of a main part of an image forming apparatus 1C according to the present embodiment. FIG. 12B is a bottom view of a sensor unit 10C illustrated in FIG. 12A. As illustrated in FIGS. 12A and 12B, the sensor unit 10C differs from the sensor unit 10 in that the light-emitting units 11 and 12 include filters 16 and 17, respectively.

The filter 16 transmits the light in a band containing the peak wavelength of the emission light L1 and narrower than that of the emission light L1. Likewise, the filter 17 transmits the light in a band containing the peak wavelength of the emission light L2 and narrower than that of the emission light L2. The band of each of the emission light L1 and the emission light L2 refers to the range of the wavelength between the two points whose intensities of the light are 50 when the intensity of the light at the peak wavelength of intensity spectrum of the emission light is assumed to be 100. The band of the light that the filters 16 and 17 transmit refers to the range of the wavelength between the two points whose intensities of the transmitted light are 50 when the intensity of the transmitted light at the peak wavelength of the transmission spectrum of each filter is assumed to be 100. To put it differently, the width of the above-mentioned band is a half-value width of the peak.

In the image forming apparatus 1C, the light that has passed through the filters 16 and 17 is incident on the paper 100, and the reflected light R1 and R2 is received by the light-receiving unit 13. Hence, the received-light intensities of the reflected light R1 and R2 having a narrow wavelength half-value width can be measured. Accordingly, the accuracy of the measurement can be improved.

Note that the filters may be included in the light-receiving unit 13, instead of in the light-emitting units 11 and 12. In this case, the filters included in the light-receiving unit 13 may transmit the following two kinds of light.

(1) Light in a band containing the peak wavelength of the emission light L1

(2) Light in a band containing the peak wavelength of the emission light L2.

In a case where the filters included in the light-receiving unit 13 is configured to narrow the wavelength bands of the emission light L1 and L2, as in the case where the light-emitting units 11 and 12 include the filters 16 and 17, the received-light intensities of the reflected light R1 and R2 having the narrow half-value width can be measured. The filters included in the light-receiving unit 13 need not narrow the wavelength bands of the emission light L1 and L2. With filters configured not to narrow the wavelength bands of the emission light L1 and L2, in a case that the filters can block the other light incident on the light-receiving unit 13 than the emission light L1 and L2 (e.g., the indoor light and the sun light that leak into the image forming apparatus 1C from the outside), the measurement accuracy of the reflected light R1 and R2 can be improved.

### Fifth Embodiment

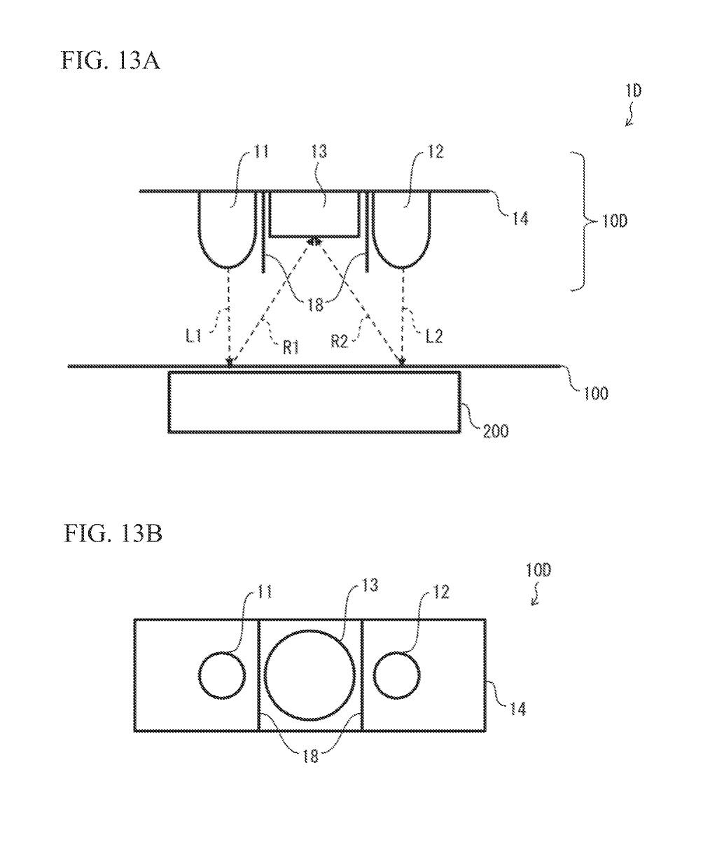

Another embodiment of the present invention will be described below with reference to FIGS. 13A and 13B.

FIG. 13A is a diagram illustrating a configuration of a main part of an image forming apparatus 1D according to the present embodiment. FIG. 13B is a bottom view of a sensor unit 10D illustrated in FIG. 13A. As illustrated in FIGS. 13A and 13B, the sensor unit 10D differs from the sensor unit 10 in that the sensor unit 10D includes a light blocking part 18.

In the sensor unit 10D, the light blocking part 18 blocks the light emitted by the light-emitting units 11 and 12 directly towards the light-receiving unit 13. Hence, the determination accuracy of the moisture content and the paper type of the paper 100 can be improved.

As illustrated in, for example, FIGS. 13A and 13B, the light blocking part 18 may be a flat-plate-shaped member disposed between the light-receiving unit 13 and each of the light-emitting units 11 and 12. Alternatively, the light blocking part 18 may be a dome-shaped member configured to cover a portion of the light-receiving unit 13.

### Implementation Example by Software

The control blocks (in particular, the light-emission control unit 41, the absorbance calculation unit 42, and the paper determination unit 43) of the image forming apparatuses 1, 1X, 1A, 1B, 1C, and 1D may be implemented by a logic circuit (hardware) formed in an integrated circuit (IC chip) and the like, or may be implemented by software using a Central Processing Unit (CPU).

In the latter case, the image forming apparatuses 1, 1X, 1A, 1B, 1C, and 1D includes CPU configured to execute a command of a program, that is software, to realize each function, Read Only Memory (ROM) or a storage device (these are referred to as “recording medium”) configured to store the program and various types of data in a manner capable of being read by a computer (or CPU), Random Access Memory (RAM) to develop the program, and the like. Then, the computer (or CPU) reads the program from the recording medium and executes the program to achieve the object of an aspect of the present invention. As the recording medium, a “non-transitory tangible medium”, such as a tape, a disk, a card, a semiconductor memory, and a programmable logic circuit may be used. Further, the program may be supplied to the computer via any transmission medium (a communication network, a broadcast wave, or the like) able to transmit the program. Note that an aspect of the present invention may be implemented in a form of data signal embedded in a carrier wave, which is embodied by electronic transmission of the program.

### Supplement

An image forming apparatus (1) according to a first aspect of the present invention includes: an illuminating unit (light-emitting units 11, 12) configured to illuminate paper (100) with a plurality of types of emission light (L1, L2) having mutually different peak wavelengths; a light-receiving unit (13) configured to receive a plurality of types of reflected light (R1, R2), reflected from the paper, of the plurality of types of emission light; and a determination unit (paper determination unit 43) configured to determine a moisture content and a paper type of the paper, based on received-light intensities of the plurality of types of reflected light received by the tight-receiving unit.

With the configuration described above, the plurality of types of emission light having mutually different peak wavelengths are emitted from the illuminating unit to the paper. The plurality of types of reflected light, reflected from the paper, of the plurality of types of emission light are received by the light-receiving unit. On the basis of the received-light intensities of the reflected light, the determination unit determines the received-light intensities for the paper and the paper type. This simplifies the sensor structure, and both the paper type and the moisture content of the paper can also be determined.

The image forming apparatus according to a second aspect of the present invention is the image forming apparatus of the first aspect. The light-receiving unit may preferably be a single unit.

With the configuration described above, the sensor structure can be further simplified.

The image forming apparatus according to a third aspect of the present invention is the image forming apparatus of the first aspect or the second aspect and further includes a calculation unit (absorbance calculation unit 42) configured to calculate an absorbance of the paper, based on a ratio between the received-light intensities of the plurality of types of reflected light received by the light-receiving unit and a reference received-light intensity. The determination unit may be configured to determine the moisture content and the paper type, based on the absorbance calculated by the calculation unit.

With the configuration described above, the calculation unit calculates the absorbance of the paper, based on a ratio between each of the received-light intensities of the plurality of types of reflected light having a plurality of types of wavelengths and the reference received-light intensity. Then the determination unit determines the moisture content and the paper type of the paper on the basis of the absorbance The use of the ratio allows errors in the amount of light emitted from the illuminating unit, the amplification rates of the received signals, or the like to be canceled. This can eliminate the effect of the errors.

The image forming apparatus according to a fourth aspect of the present invention is the image forming apparatus of the third aspect. The determination unit may preferably be configured to determine the moisture content with a prescribed function prepared beforehand for a prescribed paper type and indicating a changing absorbance depending on the moisture content.

With the configuration described above, the moisture content of the paper can be determined easily.

The image forming apparatus according to a fifth aspect of the present invention is the image forming apparatus of the fourth aspect. The determination unit may preferably be configured to determine the paper type with a plurality of the prescribed functions prepared beforehand for each of a plurality of prescribed paper types.

With the configuration described above, the paper type can also be determined easily.

The image forming apparatus according to a sixth aspect of the present invention is the image forming apparatus of the first aspect and further includes a calculation unit, wherein the illuminating unit (light-emitting unit 11, 12, and 15) may be configured to illuminate the paper with at least three types of emission light (L1, L2, L5) having mutually different peak wavelengths, the calculation unit may be configured to calculate an absorbance of the paper, based on a ratio between a received-light intensity of reflected light, reflected from the paper, of one type of emission light of the at least three types of emission light and received-light intensities of reflected light, reflected from the paper, of the remaining two or more types of emission light. The determination unit may be configured to determine the moisture content and the paper type, based on the absorbance calculated by the calculation unit.

With the configuration described above, the moisture content and the paper type of the paper are determined on the basis of the ratio between: of the at least three types of reflected light, the received-light intensity of one type of reflected light and the received-light intensities of the remaining two or more types of reflected light. Hence, the moisture content and the paper type of the paper can be determined by solely measuring the received-light intensities for the paper.

The image forming apparatus according to a seventh aspect of the present invention is any one of the image forming apparatuses of the first to the sixth aspects. An optical axis of the plurality of types of emission light emitted by the illuminating unit or a normal line to a light-receiving surface of the light-receiving unit may preferably make a not-perpendicular angle with the paper, and the light-receiving unit may preferably be configured to receive the plurality of types of reflected light reflected regularly from the paper.

With the configuration described above, the light-receiving unit can receive the reflected light efficiently in a case where the regularly reflected component of the reflected light has a high intensity.

The image forming apparatus according to an eighth aspect of the present invention is the image forming apparatus of any one of the first to the seventh aspects. The image forming apparatus may preferably include a filter (16, 17) configured to transmit light in a band narrower than a band of one of the plurality of types of emission light, and the band may contain a peak wavelength of the one of the plurality of types of emission light emitted from the illuminating unit.

With the configuration described above, the illuminating unit emits light having a prescribed wavelength containing the peak wavelength of the emission light. Hence, the illuminating unit is a light source configured to emit emission light having a narrow wavelength half-value width.

The image forming apparatus according to a ninth aspect of the present invention is the image forming apparatus of any one of the first to the eighth aspect and may preferably include a light blocking part (18) configured to block the plurality of types of emission light emitted by the illuminating unit directly towards the light-receiving unit.

With the configuration described above, since the emission light that is emitted by the illuminating unit directly towards the light-receiving unit is blocked, the determination accuracy of the moisture content and the paper type of the paper can be improved.

The image forming apparatus according to a tenth aspect of the present invention is the image forming apparatus of any one of the first to the ninth aspect. The plurality of types of emission light may preferably have respective peak wavelengths that are different from each other by at least 10 nm. With the configuration described above, the paper type and the moisture content can be determined appropriately.

The image forming apparatus according to an eleventh aspect of the present invention is the image forming apparatus of the first to the tenth aspects. The image forming apparatus may preferably be configured to form an image on the paper in accordance with an image forming condition set, based on a determination result by the determination unit. With the configuration described above, an image can be formed on the paper in accordance with appropriate image forming conditions.

A determination method according to a twelfth aspect of the present invention is a method of determining a moisture content and a paper type of paper (100), the method including: making an illuminating unit (light-emitting units 11, 12) illuminate the paper with a plurality of types of emission light (L1, L2) having mutually different peak wavelengths; making a light-receiving unit (13) receive a plurality of types of reflected light (R1, R2), reflected from the paper, of the plurality of types of emission light; and determining the moisture content and the paper type of the paper, based on received-light intensities of the plurality of types of reflected light. With the configuration described above, effects similar to those in the first aspect can be obtained.

An aspect of the present invention is not limited to each of the above-described embodiments. It is possible to make various modifications within the scope of the claims. An embodiment obtained by appropriately combining technical elements each disclosed in different embodiments falls also within the technical scope of the aspect of the present invention. Furthermore, technical elements disclosed in the respective embodiments may be combined to provide a new technical feature.

### Other Ways of Expressing Aspect of Invention

An aspect of the present invention can be expressed in the following ways. Specifically, an image forming apparatus according to an aspect of the present invention includes: a sensor unit including a light-emitting unit configured to illuminate paper with light having different wavelengths and a single light-receiving unit configured to receive reflected light reflected from the paper; and a control unit configured to determine a moisture content and a paper type of the paper on the basis of a received-light intensity.

In the image forming apparatus according to an aspect of the present invention, the control unit is configured: to obtain absorbance of the paper for each of the plurality of wavelengths on the basis of a ratio between a received-light intensity for the paper and a received-light intensity for a reference plate, these received-light intensities being measured under the equivalent conditions; and to determine the moisture content and the paper type of the paper on the basis of the absorbance.

In addition, in the image forming apparatus according to an aspect of the present invention, the light-emitting unit is configured to emit light having at least three different wavelengths on the paper, and the control unit is configured to determine the moisture content and the paper type of the paper on the basis of a ratio between the intensity of the received light having any one of the wavelengths and the intensity of the received light having the remaining ones of the wavelengths.

In addition, in the image forming apparatus according to an aspect of the present invention, the light-emitting unit or the light-receiving unit is angled with respect to the paper.

In addition, in the image forming apparatus according to an aspect of the present invention, the sensor unit includes a wavelength filter configured to transmit light having a prescribed wavelength of the light emitted by the light-emitting unit.

In addition, in the image forming apparatus according to an aspect of the present invention, the sensor unit includes a light-blocking plate configured to block light emitted by the light-emitting unit and incident directly on the light-receiving unit.

### CROSS-REFERENCE TO RELATED APPLICATIONS

This application claims the benefit of priority to Japanese Patent Application Number 2016-158159 filed on Aug. 10, 2016. The entire contents of the above-identified application are hereby incorporated by reference.

### REFERENCE SIGNS LIST

1, 1A, 1B, 1C, 1D, and 1X image formation apparatus
11, 12, 15 Light-emitting unit (illuminating unit)
13 Light-receiving unit
16, 17 Filter
18 Light blocking part
42 Absorbance calculation unit (calculation unit)
43 Paper determination unit (determination unit)
100 Paper
L1, L2, L5 Emission light
R1, R2, R5 Reflected light

1. An image forming apparatus comprising:
an illuminating unit configured to illuminate paper with a plurality of types of emission light having mutually different peak wavelengths;
a light-receiving unit configured to receive a plurality of types of reflected light, reflected from the paper, of the plurality of types of emission light; and
a determination unit configured to determine a moisture content and a paper type of the paper, based on received-light intensities of the plurality of types of reflected light received by the light-receiving unit.

2. The image forming apparatus according to claim 1,
wherein the light-receiving unit is a single unit.

3. The image forming apparatus according to claim 1 further comprising:
a calculation unit configured to calculate an absorbance of the paper, based on a ratio between the received-light intensities of the plurality of types of reflected light received by the light-receiving unit and a reference received-light intensity,
wherein the determination unit is configured to determine the moisture content and the paper type, based on the absorbance calculated by the calculation unit.

4. The image forming apparatus according to claim 3,
wherein the determination unit is configured to determine the moisture content with a prescribed function prepared beforehand for a prescribed paper type and indicating a changing absorbance depending on the moisture content.

5. The image forming apparatus according to claim 4,
wherein the determination unit is configured to determine the paper type with a plurality of the prescribed functions provided beforehand for each of a plurality of prescribed paper types.

6. The image forming apparatus according to claim 1, further comprising:
a calculation unit, wherein the illuminating unit is configured to illuminate the paper with at least three types of emission light having mutually different peak wavelengths, the calculation unit is configured to calculate an absorbance of the paper, based on a ratio between a received-light intensity of reflected light, reflected from the paper, of one type of emission light of the at least three types of emission light and received-light intensities of reflected light, reflected from the paper, of the remaining two or more types of emission light,
wherein the determination unit is configured to determine the moisture content and the paper type, based on the absorbance calculated by the calculation unit.

7. The image forming apparatus according to claim 1,
wherein an optical axis of the plurality of types of emission light emitted by the illuminating unit or a normal line to a light-receiving surface of the light-receiving unit makes a not-perpendicular angle with the paper; and
the light-receiving unit is configured to receive the plurality of types of reflected lights reflected regularly from the paper.

8. The image forming apparatus according to claim 1, further comprising:
a filter configured to transmit light in a band containing a peak wavelength of one of the plurality of types of emission light and narrower than a band of the one of the plurality of types of emission light.

9. The image forming apparatus according to claim 1, further comprising:
a light blocking part configured to block the plurality of types of emission light emitted by the illuminating unit directly towards the light-receiving unit.

10. The image forming apparatus according to claim 1,
wherein the plurality of types of emission light have respective peak wavelengths that are different from each other by at least 10 nm.

11. The image forming apparatus according to claim 1,
wherein the image forming apparatus is configured to form an image on the paper in accordance with an image forming condition set, based on a determination result by the determination unit.

12. A method for determining a moisture content and a paper type of paper, the method comprising:
making an illuminating unit illuminate the paper with a plurality of types of emission light having mutually different peak wavelengths;
making a light-receiving unit receive a plurality of types of reflected light, reflected from the paper, of the plurality of types of emission light; and
determining the moisture content and the paper type of the paper, based on received-light intensities of the plurality of types of reflected light.
